(19) United States
(12) Patent Application Publication
(10) Pub. No.: US 2021/0177889 A1
(43) Pub. Date: Jun. 17, 2021

(54) GASEOUS PHARMACEUTICAL COMPOSITION FOR HYPERTENSION THERAPY

(71) Applicants: KEIO UNIVERSITY, Tokyo (JP); TAIYO NIPPON SANSO CORPORATION, Tokyo (JP)

(72) Inventors: Motoaki Sano, Tokyo (JP); Tomoyoshi Tamura, Tokyo (JP); Eiji Kobayashi, Tokyo (JP)

(21) Appl. No.: 17/101,162

(22) Filed: Nov. 23, 2020

(30) Foreign Application Priority Data
Dec. 16, 2019 (JP) 2019-226366

(51) Int. Cl. A61K 33/00; A61P 9/12; A61K 9/00; A61M 16/10; A61M 16/12

(52) U.S. Cl. CPC A61K 33/00; A61P 9/12; A61K 9/007; A61M 2202/0266; A61M 16/12; A61M 2202/02; A61M 16/10

(57) ABSTRACT

The object of the invention is to provide a novel pharmaceutical composition for hypertension therapy that can be applied to a wide range of hypertensive patients.

According to the present disclosure, a gaseous pharmaceutical composition for improving hypertension comprising hydrogen gas is provided. The pharmaceutical composition according to the present disclosure can be used for improvement of a wide range of hypertension including essential hypertension.

### TECHNICAL FIELD

A part of the content of the present application has been published within 1 year retroactively from the effective filing date of the present application (Dec. 16, 2019) by the inventor of the present application or a joint inventor thereof, or by a third party who had directly learned the subject matter of the present invention from the inventor of the present application at the request of the applicant of the present application, respectively, in the following URL (https://kaken.nii.ac.jp/report/KAKENHI-PROJECT-16K11420/16K114202017hokoku/; https://kaken.nii.ac.jp/grant/KAKENHI-PROJECT-16K11420/) (Publication date: Dec. 17, 2018); the Proceedings of the 9th annual conference of Japanese Biomedical Society for Molecular Hydrogen (Publication date: Aug. 31, 2019), and the 9th annual conference of Japanese Biomedical Society for Molecular Hydrogen (Publication date: Sep. 1, 2019).

The present disclosure relates to a pharmaceutical composition for hypertension therapy, more specifically to a gaseous pharmaceutical composition for hypertension therapy comprising hydrogen gas.

### BACKGROUND ART

The global hypertension prevalence in adults is estimated to be 1.13 billion people. Hypertension is a powerful risk factor of cardiovascular disease (CVD) and chronic kidney disease (CKD), and is onset in 80V or more of CKD patients. Since CKD promotes hypertension, and this possibly contributes to the progression of CKD, it is important to optimize the care at the predialysis stage of CKD. In this way, even though emphasis is placed on hypertension therapy, blood pressure control is insufficient in 60% or more of hypertensive patients, and target blood pressure therapy has not been achieved.

Molecular hydrogen (H2) is a pluripotent gas having antioxidative property and anti-inflammatory property, and thus far has been a promising therapeutic option of ischemia-reperfusion injury in urgent and critical care settings of acute myocardial infarction, cardiac arrest, hemorrhagic shock, and the like in animal models (Patent Literature 1 and Non-Patent Literatures 1-5).

In regard to hypertension as well, the therapeutic effect of hydrogen has been suggested in terminal stage renal disease patients thus far (Non-Patent Literatures 6 and 7). Specifically, it was reported that hypertension after dialysis was improved compared to conventional hemodialysis in a chronic hemodialysis patient dialyzed by a hydrogen concentrated dialysate manufactured by mixing reverse osmosis water comprising dissolved hydrogen produced by water electrolysis with a dialysate concentrate.

### CITATION LIST

[Patent Literature 1] International Publication No. 2018/021175
[Non-Patent Literature 1] Journal of the American Heart Association 2012; doi:10.1161/JAHA.112.003459; Circulation. 2014 Dec. 9; 130(24):2173-80
[Non-Patent Literature 2] Hayashida, K. et al. Inhalation of hydrogen gas reduces infarct size in the rat model of myocardial ischemia-reperfusion injury. Biochem. Biophys. Res. Commun. 373, 30-35; 10.1016/j.bbrc.2008.05.165 (2008)
[Non-Patent Literature 3] Hayashida, K. et al. H (2) gas improves functional outcome after cardiac arrest to an extent comparable to therapeutic hypothermia in a rat model. J. Am. Heart Assoc. 1, e003459; 10.1161/JAHA.112.003459 (2012).
[Non-Patent Literature 4] Hayashida, K. et al. Hydrogen inhalation during normoxic resuscitation improves neurological outcome in a rat model of cardiac arrest independently of targeted temperature management. Circulation 130, 2173-2180; 10.1161/CIRCULATIONAHA.114.011848 (2014).
[Non-Patent Literature 5] Matsuoka, T. et al. Hydrogen gas inhalation inhibits progression to the “irreversible” stage of shock after severe hemorrhage in rats. J. Trauma Acute Care Surg. 83, 469-475; 10.1097/TA.0000000000001620 (2017).
[Non-Patent Literature 6] Nakayama, M. et al. A novel bioactive haemodialysis system using dissolved dihydrogen (H2) produced by water electrolysis: a clinical trial. Nephrol. Dial. Transplant. 25, 3026-3033; 10.1093/ndt/gfq196 (2010))
[Non-Patent Literature 7] Nakayama, M. et al. Novel haemodialysis (HD) treatment employing molecular hydrogen (H2)-enriched dialysis solution improves prognosis of chronic dialysis patients: A prospective observational study. Sci. Rep. 8, 254; 10.1038/s41598-017-18537-x (2018)

### SUMMARY OF THE INVENTION

### Problems to be Solved by the Invention

However, conventional technology assumes use in dialysis in the form where hydrogen is dissolved in the dialysate, and it cannot be applied to hypertensive patients (such as CKD patients) before dialysis or hypertensive patients not attributed to kidney dysfunction.

Accordingly, the object of the invention is to provide a novel pharmaceutical composition for hypertension therapy that can be applied to a wide range of hypertensive patients including subjects having essential hypertension, that does not depend on a particular cause.

### Means for Solving the Problems

As a result of repeated investigation by the present inventors to solve the above problem, it was found for the first time that hypertension in a hypertensive subject including hereditary hypertension can be improved by administering hydrogen in gaseous form (hydrogen) to the hypertensive subject.

In other words, the present disclosure encompasses the following characteristics.

[1] A gaseous pharmaceutical composition for improving hypertension, characterized in that it comprises hydrogen gas.

[2] The pharmaceutical composition according to [1], wherein the improvement of said hypertension comprises the improvement of diastolic hypertension.

[3] The pharmaceutical composition according to [2], wherein a subject having said hypertension is a subject having a diastolic blood pressure of 100 mmHg or higher.

[4] The pharmaceutical composition according to any one of [1] to [3], wherein said hypertension is essential hypertension or secondary hypertension.

[5] The pharmaceutical composition according to [4], wherein said hypertension is essential hypertension.

[6] The pharmaceutical composition according to [4], wherein said hypertension is secondary hypertension.

[7] The pharmaceutical composition according to [6], wherein said secondary hypertension is hypertension attributed to renal dysfunction.

[8] The pharmaceutical composition according to [7], wherein said kidney dysfunction is attributed to renal disease or attributed to nephrectomy or partial nephrectomy.

[9] The pharmaceutical composition according to any one of [6] to [8], wherein a subject having said secondary hypertension is a subject that has not received dialysis.

[10] The pharmaceutical composition according to any one of [1] to [9], characterized in that said pharmaceutical composition further comprises oxygen gas.

[11] The pharmaceutical composition according to any one of [1] to [10], characterized in that said pharmaceutical composition further comprises inert gas.

[12] The pharmaceutical composition according to any one of [1] to [11], characterized in that said hydrogen concentration in said pharmaceutical composition is 0.1%-4.0% (v/v).

[13] The pharmaceutical composition according to any one of [1] to [12], characterized in that said hydrogen concentration in said pharmaceutical composition is 1.0%-2.0% (v/v).

[14] The pharmaceutical composition according to any one of [1] to [13], characterized in that said hydrogen gas is provided by a hydrogen gas generator or a container containing hydrogen gas.

[15] The pharmaceutical composition according to [14], characterized in that said hydrogen gas generator is equipped with a hydrogen production means by water electrolysis.

[16] The pharmaceutical composition according to [14], characterized in that said container contains a mixed gas of hydrogen gas and nitrogen gas.

[17] The pharmaceutical composition according to any one of [14] to [16], characterized in that said hydrogen gas generator or said container is further equipped with a control means for monitoring and adjusting the amount of hydrogen gas supplied to the subject on said device or container itself or on a piping connected to said device or container.

[18] A method for hypertension therapy comprising administering hydrogen gas to a subject having hypertension.

An invention of any combination of one or more of the aspects listed above are also encompassed by the scope of the present invention.

### Effects of the Invention

According to the present disclosure, a novel pharmaceutical composition for hypertension therapy that can be applied to a wide range of hypertensive patients is provided.

### BRIEF DESCRIPTION OF THE DRAWINGS

FIG. 1 shows the summary of each experimental design employed in the Examples of the present application. In the figures, each abbreviation and symbol respectively indicates the following: white arrow, 5/6 nephrectomy; black arrow, implantation of telemetry transmitter; gray triangle, invasive hemodynamics monitoring by femoral artery catheter; black triangle, heart rate variability analysis; white triangle, non-invasive hemodynamics measurement by tail-cuff method, gray bar, recording of hemodynamics daily telemetry; LEW, Lewis rat; SHR, spontaneous hypertension rat.

FIG. 2 shows the influence of H₂ inhalation at the early post-partial nephrectomy period on the hemodynamics and renal function at 4 weeks. In the figures, each abbreviation respectively indicates the following: MAP, mean arterial pressure; BP, blood pressure. Each group is N=3, and data is represented as mean±SE: *P<0.05, **P<0.01 (analysis of variance).

FIG. 3 shows the influence of H₂ inhalation at the early post-partial nephrectomy period on walking blood pressure and renal function. Abbreviations in the figures indicate the following: MAP, mean arterial pressure. Each group is N=3, and data is represented as mean±SE: *P=0.04 (mixed effects model).

FIG. 4 shows the effect of delayed H₂ inhalation on hemodynamics in nephrectomized rats. In the figures, each abbreviation respectively indicates the following: BL, baseline; BP, blood pressure; bpm, beats per minute. Each group for blood pressure measurement is N=17, and data is represented as mean±SE.

FIG. 5 shows the effect of H_{z} on blood pressure fluctuation. Abbreviations in the figures indicate the following: LEW, Lewis rat; TTI, telemetry transmitter implantation; ΔLFₙᵤ, low-frequency power in normalised units; ΔHFₙᵤ, high-frequency power in normalized units.

FIG. 6 shows the hemodynamics effect of H₂ inhalation in spontaneous hypertension rats. In the figures, each abbreviation and symbol respectively indicates the following: bidirectional arrow, duration of intermittent gas inhalation; AUC, area under the curve; bpm, beats per minute; h, hour; HR, heart rate; MAP, mean arterial pressure. For data, each group is N=3, and data is represented as mean±SE: *P=0.04 (paired t-test).

FIG. 7 shows the hemodynamics effect of H inhalation in spontaneous hypertension rats. In the figures, each abbreviation respectively indicates the following: HF, high frequency; LF, low frequency; HFₙᵤ, normalized high frequency; LF, normalized low frequency. For data, each group is N=3, and data is represented as mean±SE: *P=0.04 (paired t-test).

### DESCRIPTION OF EMBODIMENTS

The present disclosure relates to a pharmaceutical composition for improving hypertension (hereinafter also referred to as the pharmaceutical composition according to the present disclosure).

Hypertension refers to a pathology where the blood pressure is constantly high. Specifically, upon repeated measurement of blood pressure, hypertension is diagnosed if the systolic blood pressure is higher than the reference value 140 mmHg at any measurement point (also referred to herein as “systolic hypertension”) and/or the diastolic blood pressure is higher than the reference value 90 mmHg (also referred to herein as “diastolic hypertension”).

“Improvement of hypertension” herein refers to reducing at least either one of systolic hypertension or diastolic hypertension to said reference value or lower over at least a certain period of time during or after administration of the pharmaceutical composition according to the present disclosure.

In another embodiment of the present disclosure, “improvement of hypertension” comprises at least improving diastolic hypertension over at least a certain period of time during or after administration of the pharmaceutical composition according to the present disclosure.

Said “at least a certain period of time” herein may vary depending on the severity of symptoms in the subject, age, sex, and the administration concentration and administration duration of the pharmaceutical composition according to the present disclosure. For example, said “at least a certain period of time” may be at least 12 hours, preferably at least 24 hours, more preferably at least 48 hours, more preferably at least 72 hours, more preferably at least 96 hours, more preferably at least 120 hours, more preferably at least 144 hours, and more preferably at least 168 hours from the end of administration of the pharmaceutical composition of the present invention.

Without being bound to any theory, according to the pharmaceutical composition according to the present disclosure, it is thought that the sympathetic nerve function is suppressed in the administered subject and the activation of the parasympathetic nerve is promoted due to hydrogen gas, and thus hypertension is improved.

The pharmaceutical composition according to the present disclosure is characterized in that it is a gaseous pharmaceutical composition comprising hydrogen gas. Moreover, since the pharmaceutical composition according to the present disclosure is a gaseous pharmaceutical composition, it is characterized in that it is continuously administered to a subject over a certain period of time. In the present disclosure, the hydrogen atom may be any of all of its isotopes, i.e., protium (P or ¹H), deuterium (D or ²H), and tritium (T or ³H). Accordingly, P₂, PD, PT, DT, D₂, and T₂ may be comprised as molecular hydrogen. In a preferred aspect of the present disclosure, 99% or more of the hydrogen gas comprised in the pharmaceutical composition according to the present disclosure is natural molecular hydrogen, P₂.

The pharmaceutical composition according to the present disclosure can further comprise oxygen gas. The oxygen gas may be mixed in advance with hydrogen gas and exist in the form of a mixed gas, or may be mixed with hydrogen gas immediately before or at the time of administration to a subject.

The pharmaceutical composition according to the present disclosure can further comprise inert gas. Inert gas is used with the objective of explosion protection and concentration adjustment of the hydrogen or oxygen gas, and accordingly may exist in the form of a mixed gas with hydrogen and/or oxygen gas. As inert gas that can be used in the pharmaceutical composition according to the present disclosure, although not limited thereto, nitrogen gas, helium gas, argon gas, and the like can be used. In one embodiment of the present disclosure, inexpensive nitrogen gas is used as the inert gas.

In the pharmaceutical composition according to the present disclosure, the concentration range of the hydrogen gas can be, but is not limited to, e.g. any concentration between 0.1-4.0% (v/v). The lower limit of the hydrogen gas concentration is set as the lower limit of the concentration that allows exertion of the effect of improving hypertension. Accordingly, the minimum concentration that can improve hypertension can be appropriately set depending on the severity of the subject, the presence or absence of a disease to be the cause of hypertension, sex, age, and the like. In one embodiment, the lower limit of the hydrogen gas can be selected from between 0.1-1.0%, such as 0.5%. On the other hand, the upper limit of the hydrogen gas concentration is set in regard to safety, since the lower explosion limit of hydrogen in air is 4%. Accordingly, the upper limit of the hydrogen gas can be selected from any concentration at 4% or lower, such as 3.0%, 2.5%, or 2.0%, and the like, as long as safety is secured.

In the pharmaceutical composition according to the present disclosure, the concentration of the oxygen gas can be, when the hydrogen gas concentration is assumed to be 0.1-4.0% (v/v), in the range of 21%-99.9% (v/v).

In the pharmaceutical composition according to the present disclosure, the concentration of the inert gas is set in a range that appropriately maintains the concentration of the hydrogen and/or oxygen gas, as well as affords explosion protection effect of these gases. Accordingly, for the concentration of the inert gas, those skilled in the art can appropriately set an appropriate concentration depending on the concentration of the hydrogen and/or oxygen gas used. Such concentration of inert gas may be, for example when the inert gas is a nitrogen gas, e.g. arbitrary taken in the range of 0-78.9% (v/v).

Note that the concentration of gas used throughout the present specification is the content at 20° C. and 101.3 kPa.

The pharmaceutical composition according to the present disclosure may further comprise other atmospheric gas such as carbon dioxide, air, or anesthetic gas, and the like, as long as it does not compromise the effect due to hydrogen gas.

The pharmaceutical composition according to the present disclosure can be administered to a subject by e.g. inhalation employing an inhalation means. Such inhalation means can include, but is not limited to, e.g. an inhaler mask. It is preferred that the inhaler mask simultaneously covers the mouth and the nose of the subject so that administration to a subject at an appropriate concentration is realized.

In one embodiment of the present disclosure, the pharmaceutical composition according to the present disclosure is provided in a form that may be administered to the subject as-is.

For example, as an example, in this embodiment, the pharmaceutical composition according to the present disclosure is provided in the form of a mixed gas that is prepared by mixing hydrogen gas and inert gas in advance, as well as oxygen gas for breathing and any other gas at appropriate concentrations.

In another aspect of the present disclosure, the pharmaceutical composition according to the present disclosure is provided in a form that is prepared immediately before or at the time of administration to a subject. For example, as an example, in this embodiment, the pharmaceutical composition according to the present disclosure is provided by a container containing a mixed gas of hydrogen gas and inert gas and a container containing oxygen gas being connected to an inhaler mask via piping, and delivered to a patient at a flow rate to allow a concentration appropriate for administration to a subject. In one aspect of the present disclosure, said container may be a portable gas cylinder, as well as e.g. in the form of a large-scale storage tank that is installed outdoors. Moreover, said gas may be contained in the container in the form of compressed gas, or may be contained in a Liquid Gas Container (LGC) in liquid form such as liquid hydrogen gas. Moreover, as another example in the present disclosure, said hydrogen gas may be each supplied from a hydrogen gas generator. Such a generator can include those equipped with any hydrogen production means well-known to those skilled in the art, and such hydrogen production means can include, but is not limited to, hydrogen production means utilizing water (such as purified water, alkaline water e.g. potassium hydroxide) electrolysis, hydrogen production means utilizing hydrogen absorbing alloy (such as magnesium and vanadium), hydrogen production means utilizing heating or degassing of hydrogen-dissolved water, hydrogen production means utilizing ammonia degradation, hydrogen production means utilizing steam reforming of hydrocarbon (such as methane), hydrogen production means utilizing methanol/ethanol reforming, hydrogen production means utilizing degradation of water by a catalyst (such as titanium oxide), hydrogen production means utilizing the chemical reaction between water and metal hydride (such as alkaline earth metal hydride, alkali metal hydride, typically magnesium hydride), and the like. In the present disclosure, those equipped with hydrogen production means utilizing water electrolysis are particularly preferred.

In another aspect of the present disclosure, the pharmaceutical composition according to the present disclosure is provided by supplying hydrogen gas to a sealed chamber so that the gas concentration is maintained constant. For example, as an example, in this embodiment, the pharmaceutical composition according to the present disclosure is provided by supplying a mixed gas consisting of hydrogen gas and inert gas to a sealed chamber where a subject is present at a flow rate that maintains the hydrogen concentration in the said sealed chamber at an appropriate concentration.

The administration subject of the pharmaceutical composition according to the present disclosure is not particularly limited, and may be humans, as well as non-human mammals e.g. rodents such as mice, rats, and rabbits, monkeys, cows, horses, and goats.

In one embodiment of the present disclosure, the administration subject of the pharmaceutical composition according to the present disclosure is a human subject. Moreover, in a specific embodiment of the present disclosure, the administration subject of the pharmaceutical composition according to the present disclosure is a subject (such as a human subject) having a diastolic blood pressure of 100 mmHg or higher.

In other specific embodiments of the present disclosure, the administration subject of the pharmaceutical composition according to the present disclosure is a subject (such as a human subject) having essential hypertension. “Essential hypertension” herein shall be construed as being identical to that recognized by those skilled in the art belonging to the aforementioned technical field, and comprises all hypertension that do not have a particular disease to be the cause of hypertension. For example, essential hypertension can include, but is not limited to, familial hypertension, hypertension attributed to lifestyle, and the like.

In other specific embodiments of the present disclosure, the administration subject of the pharmaceutical composition according to the present disclosure is a subject (such as a human subject) having secondary hypertension. Disease or symptom to be the cause of secondary hypertension can include, but is not limited to, renal dysfunction and the like.

In a particular embodiment of the present disclosure, disease or symptom to be the cause of secondary hypertension is a subject (such as a human subject) having renal dysfunction. Renal dysfunction may be those attributed to any renal disease such as chronic renal failure and acute renal failure, or may be those attributed to nephrectomy or partial nephrectomy.

In yet another embodiment of the present disclosure, the administration subject of the pharmaceutical composition according to the present disclosure is a subject (such as a human subject) having renal dysfunction who has not yet been dialyzed.

The pharmaceutical composition according to the present disclosure is continuously administrated to a subject over a certain period of time. The administration time of the pharmaceutical composition according to the present disclosure is not particularly limited as long as it is a time that allows exertion of the improvement effect of hypertension by the pharmaceutical composition according to the present disclosure, and those skilled in the art can appropriately set the appropriate time depending on the severity of symptoms in the subject, age, sex, the administration concentration of the pharmaceutical composition according to the present disclosure, and the like. Such time may be, but is not limited to, for example, at least 10 minutes, at least 30 minutes, at least 1 hour, at least 2 hours, at least 3 hours, at least 4 hours, or longer.

Moreover, the administration frequency of the pharmaceutical composition according to the present disclosure is not restricted, and may be single administration or multiple administrations. For the administration interval and the administration frequency of the pharmaceutical composition according to the present disclosure, the appropriate administration interval and administration frequency can be appropriately set depending on the patient's symptoms.

The pharmaceutical composition according to the present disclosure can be provided by a device for providing the pharmaceutical composition according to the present disclosure. The device according to the present disclosure is equipped with at least a means for providing hydrogen gas, typically a container containing hydrogen gas or said hydrogen gas generator, and hydrogen gas which is the active ingredient of the pharmaceutical composition according to the present disclosure is provided by said container or hydrogen gas generator.

The device according to the present disclosure is preferably further equipped with a piping that is connected at one end with said means for providing hydrogen gas. Said piping is a hydrogen gas circulation means for delivering hydrogen gas to a gas inhalation subject, and the other end is directly connected to an inhalation means for inhaling gas, or is connected to a gas mixing device for mixing with other gases such as oxygen gas. In a preferred embodiment, a means for providing hydrogen gas is further equipped with a control means for monitoring and adjusting the amount of hydrogen gas supplied to the subject. Alternatively, it may also be a piping connected to said means for providing hydrogen gas further equipped with said control means.

The device according to the present disclosure is preferably further equipped with a gas mixing device. A gas mixing device is a means for mixing hydrogen with other gases so that the hydrogen gas from said means for providing hydrogen gas is at a concentration appropriate for administration to a subject, and it is typically connected to an oxygen gas container or an oxygen gas generator via a piping. In a preferred embodiment, said gas mixing device is further equipped with a means for monitoring and adjusting the hydrogen concentration in a mixed gas, a means for monitoring and adjusting the oxygen concentration in a mixed gas, and/or a control means for monitoring and adjusting the flow rate of a mixed gas to a subject.

In another embodiment, the device according to the present disclosure can be used in combination with an artificial ventilator. In this embodiment, said mixed gas device and an artificial ventilator are connected, and the oxygen gas sent from the artificial ventilator is mixed with hydrogen gas in the gas mixing device and enters the air intake line, or mixed with hydrogen gas introduced into the air intake line and then returns to the artificial ventilator as exhaled gas.

The terms used herein are employed for describing particular embodiments, and do not intend to limit the invention.

Moreover, the term “comprising” as used herein, unless the content clearly indicates to be understood otherwise, intends the presence of the described items (such as components, steps, elements, or numbers), and does not exclude the presence of other items (such as components, steps, elements, and numbers).

All of the disclosures of the literatures cited herein should be deemed as cited herein, and those skilled in the art can cite the related disclosed contents in these prior art literatures as a part of the present specification according to the context herein without departing from the spirit and scope of the present invention.

The present disclosure will now be described more specifically below by showing Examples, but the present disclosure is not to be limited in any way by the Examples shown below.

In this Example, it was investigated whether H₂ inhalation therapy is effective for prevention and/or therapy of hypertension in hypertensive rat model.

### EXAMPLES

### Materials and Methods

### [Animals]

Male Lewis rats (8 weeks-old, body weight 250-300 g), and 10 weeks-old male spontaneous hypertension rats (SHR/Izumo rats; hereinafter simply referred to as SHR) were used (CLEA Japan). Rats were given water and standard food ad libitum, and were not fasted before experiment. Rats were kept under a condition of standardized temperature (22±1° C.) and humidity (55±5) in a 12 hours:12 hours of light-dark cycle. Rats were acclimated to the experimental condition from at least one week before the experiment. This research was approved by institutional animal control committees (Keio University [Tokyo], No. 13002-4; and Nippon Veterinary and Life Science University [Tokyo], No. 30K-61). Rats were randomly assigned to the H₂ group or the control group.

### [Implantation of Telemetry Transmitter]

In order to monitor continuous blood pressure fluctuation, telemetry transmitters were implanted into rats. Specifically, rats were anesthetized with isoflurane, and the left groin was disinfected with 1% chlorhexidine. An incision of about 1.5 cm was made in the left groin, the left femoral artery was exposed, and a telemetry transmitter (HD-S10, Physiotel HD Telemetry, Data Science International) catheter was inserted into the left femoral artery. The tip of the transmitter catheter was placed in the abdominal aorta caudally from the branch of the renal artery. The transmitter body was inserted into a subcutaneous pocket made in the left lower back, and the skin was sutured. All surgical intervention was performed under sterile conditions.

### [Invasive and Non-Invasive Blood Pressure Measurement]

Invasive blood pressure measurement was performed 4 weeks after gas inhalation. An arterial catheter (PE50, Natsume) was inserted under isoflurane inhalation into the left femoral artery, and arterial blood pressure was measured (DX-360, Nihon Kohden).

At about 24 hours after the final gas inhalation of 5/6 nephrectomized rats, blood pressure and heart rate were non-invasively measured using the tail-cuff pressure measurement method (BP-98A, Softron). The hemodynamics parameter was measured three times, and the median was adopted as the representative value.

### [Heart Rate Variability Analysis]

Heart rate variability was analyzed using telemetry system software (Ponemah Ver. 6.3, Data Science International). Frequency domain analysis (sampling rate of 500 Hz; very low frequency (VLF) (0.05-0.25 Hz); low frequency (LF) (0.25-1.0 Hz); and high frequency (HF) (1.0-3.0 Hz)) was carried out using the arterial pressure waveform data of the first one minute of every five minutes (about 300 to 400 beats). In heart rate variability analysis, data recorded in the first 15 minutes was excluded, and effective data in the first one minute out of a block of every 5 minutes was used. LF and HF each reflected the sympathetic nerve component and the parasympathetic nerve component (Akselrod, S. et al. Power spectrum analysis of heart rate fluctuation: a quantitative probe of beat-to-beat cardiovascular control. Science 213, 220-222 (1981); Pagani, M. et al. Power spectral analysis of heart rate and arterial pressure variabilities as a marker of sympatho-vagal interaction in man and conscious dog. Circ. Res. 59, 178-193 (1986); and Montano, N. et al. Power spectrum analysis of heart rate variability to assess the changes in sympathovagal balance during graded orthostatic tilt. Circulation 90, 1826-1831 (1994)). LF/HF ratio and HF/(LF+HF) were each handled as indicators of sympathetic nerve activity and parasympathetic nerve activity (Heart rate variability: standards of measurement, physiological interpretation and clinical use. Task Force of the European Society of Cardiology and the North American Society of Pacing and Electrophysiology. Circulation 93, 1043-1065 (1996); and Pagani, M. et al. Relationship between spectral components of cardiovascular variabilities and direct measures of muscle sympathetic nerve activity in humans. Circulation 95, 1441-1448 (1997)).

### [Production of Partial Nephrectomy Model]

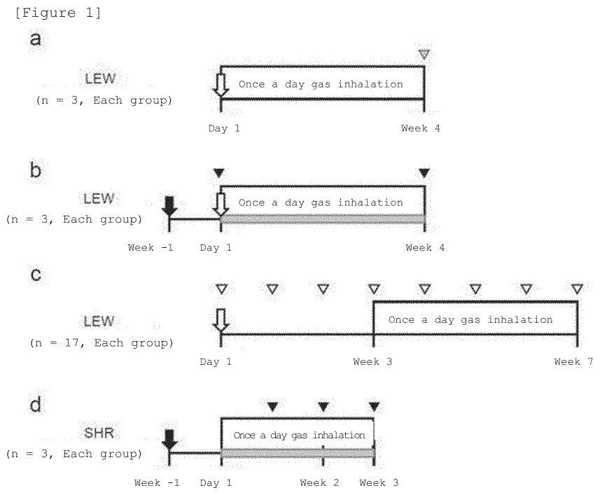

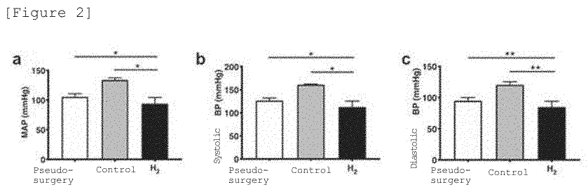

In order to elicit renal hypertension, 5/6 nephrectomy was carried out with slight modification to previously described methods (Nephrology (Carlton) 19, 552-561; 10.1111/nep.12279 (2014); J. Am. Soc. Nephrol. 4, 2023-2031 (1994); and Kidney Int. 40, 29-34; 10.1038/ki.1991.175 (1991).). Briefly, rats were anesthetized by isoflurane inhalation (induction at 4% and maintenance at 1.5%), and upper midline laparotomy of about 4 cm was carried out. Surgical operation was performed under a microscope, the left renal artery branch was identified, and selectively ligated with 7-0 silk at a position as close as possible to the left kidney so that macroscopic infarction is caused in about ⅔ of the left renal cortex. In the first pilot research (FIGS. 1a and 2), a high-temperature cautery pen (AA11, Bovie, Tenn., U.S.A.) was used to add the resection of the infarcted portion of the kidney. Subsequently, the right renal artery, the renal vein, and the urinary duct was ligated with 4-0 silk, and the right kidney were resected. The abdominal wall and the skin were closed with a 4-0 nylon thread. The anesthetic time was uniformly 30 minutes in all rats.

### [Gas Inhalation]

H₂ gas (1.3% H₂+21% O₂+77.7 N₂) and control gas (21% O₂+79% N₂) were filled into gas cylinders in advance at a factory. The H₂ group and the control group were allowed to inhale the H₂ gas and the control gas that were mixed in advance. All animals were allowed to inhale the assigned gas at 10 L/minute for 1 hour in an established anesthesia box, and inhaled for an additional three minutes to purge the box (i.e. a total of 63 minutes). In the nephrectomy model and SHR, gas inhalation was repeated every day for 4 weeks and 2 weeks, respectively.

### [Statistical Analysis]

Descriptive statistics was represented as mean±standard error of the mean. Comparison was performed by appropriately using analysis of variance, unpaired t-test, paired t-test, or Mann-Whitney U-test. Using the mixed effects model, repeated measurement variables were analyzed. All tests were two-tailed, and P value<0.05 was considered statistically significant. All statistical analysis was carried out using GraphPad Prism 8.0 (GraphPad Software Inc.).

### Results

[Prophylactic Effect Against Hypertension Attributed to 5/6 Nephrectomy by One Hour Daily Inhalation of H₂ Gas]

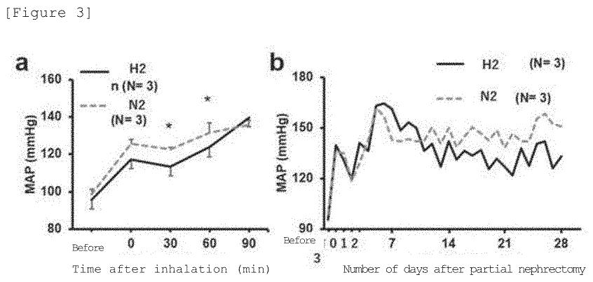

In order to evaluate the hypotensive effect of inhaled H₂, Lewis rats (8 weeks-old; each group N=3) were allowed to inhale 1.3% H₂ or control gas immediately after 5/6 nephrectomy in a box with spontaneous breathing one hour daily (FIG. 1a). As indicated by a slight deviation of renal function (FIG. 3) and 100% survival rate, primary 5/6 nephrectomy established with microscopic surgery by a skilled surgeon gave reproducible results with extremely slight error. Four weeks after 5/6 nephrectomy, arterial blood pressure was significantly reduced in the H₂ group compared to the control group (mean arterial pressure (MAP) 94.2±10.3 mmHg, 134.1±3.3 mmHg, P=0.02) (FIGS. 2a-c).

In order to investigate the time course necessary for H₂ gas to exert therapeutic effect, rats implanted with telemetry (8 weeks-old; 3 for each group) were subjected to 5/6 nephrectomy, and the blood pressure fluctuation was continuously monitored (FIG. 1b). In the first gas inhalation immediately after 5/6 nephrectomy, the blood pressure of the H₂ gas group was reduced compared to the control group (117.4±1.8 mmHg vs. 125.6±4.7 mmHg, P=0.04). However, when the inhalation was stopped, the difference in blood pressure between the two groups immediately disappeared (FIG. 3a). Before the start of inhalation on Day 2, there was no difference in blood pressure between the two groups. After Week 1 of inhalation, blood pressure before inhalation began to decrease compared to the control group H; group (FIG. 3b).

[Therapeutic Effect Against Hypertension Established after 5/6 Nephrectomy by One Hour Daily Inhalation of H₂]

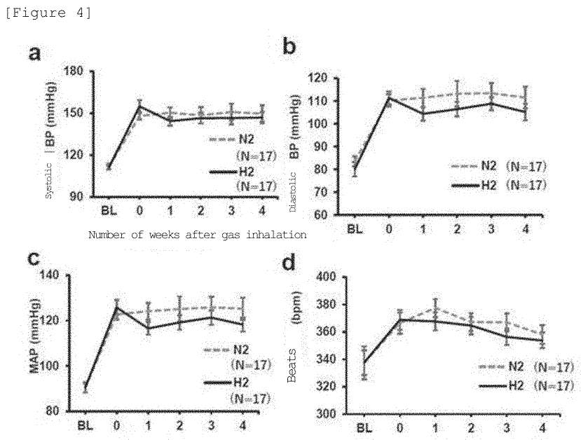

In order to evaluate the therapeutic effect of 1 hour inhalation of H₂ gas against established hypertension, Lewis rats (8 weeks-old; N=17 for each group) were subjected to 5/6 nephrectomy, and then allowed to recover for 3 weeks (FIG. 1c). Three weeks after 5/6 nephrectomy, blood pressure was equally elevated in both groups. Although there was no statistically significant difference, blood pressure began to decrease one week after starting H₂ inhalation. This hypotensive effect of H₂ gas continued through the inhalation duration (FIGS. 4a-d).

### [Effect of H₂ Inhalation on Autonomic Nervous System]

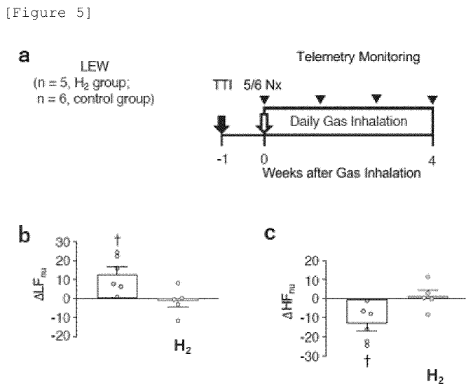

To examine the anti-hypertensive effect of H₂ in more detail, chronic and continuous monitoring of blood pressure using a non-invasive method with a wireless implantable telemetry system (FIG. 5a) was conducted. Gas inhalation was started on the day of 5/6 nephrectomy and continued for 4 weeks. Again, there was no difference in the time course of change in renal function between the two groups. A longer period of gas inhalation led to a greater decrease in blood pressure in the 5/6 nephrectomy+H₂ group, compared to the control 5/6 nephrectomy (P<0.05).

To examine the influence of H₂ therapy on autonomic nervous system activity, spectral analysis of blood pressure variability was conducted. Spectral components were obtained in normalised units (nu). Low-frequency (LF) power indicates a predominantly sympathetic tone, whereas high-frequency (HF) indicates a predominantly parasympathetic tone. Although the increase in LF power and decrease in HF power over time associated with 5/6 nephrectomy showed a tendency to be suppressed by H inhalation, the results were not significant when compared as continuous variables over a 4-week time course. In contrast, comparing the change from the baseline (day 0) to 4 weeks after starting H₂ inhalation, the increase in LF power and decrease in HF power observed in the control 5/6 nephrectomy group was significantly suppressed in the 5/6 nephrectomy+H=group (FIG. 5b, c).

### [Hemodynamics Effect of Inhaled H. Against Spontaneous Hypertension Rat]

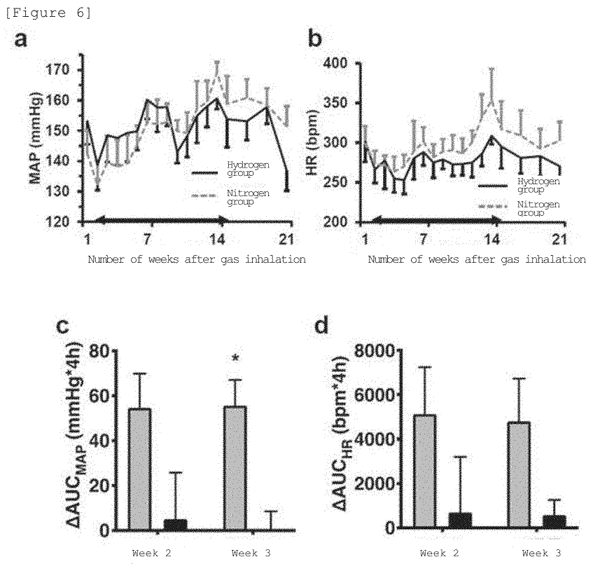

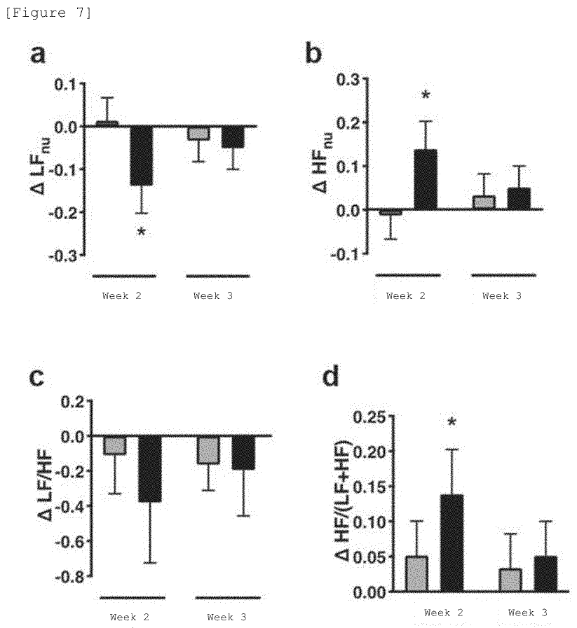

Spontaneous hypertension rats (SHR) are a well-established hereditary hypertensive model that is broadly used in hypertensive research (Okamoto, K. & Aoki, K. Development of a strain of spontaneously hypertensive rats. Jpn. Circ. J. 27, 282-293 (1963); and Rubattu, S., Struk, B., Kreutz, R., Volpe, M. & Lindpaintner, K. Animal models of genetic hypertension: what can we learn for human hypertension? Clin. Exp. Pharmacol. Physiol. 22, S386-393 (1995)). In order to investigate the consistency of the hypotensive effect of H₂ in other hypertensive models, the therapeutic effect of H₂ gas against hypertension of SHR was investigated (FIG. 1d). SHR (10 weeks-old) were randomly divided into H₂ or control groups (N=3 for each group), and the assigned gas was inhaled for 2 weeks. At 10 weeks-old, the blood pressure of SHR was already about as high as 150 mmHg in MAP. In the control group, blood pressure and heart rate continued to increase with time. However, in H₂ group, this increase in blood pressure and heart rate was suppressed in Week 2 compared to Week 1 (FIGS. 6a-d). After inhaling H₂ gas for 2 weeks, even when the inhalation of H₂ gas was stopped, blood pressure and heart rate stayed low for the next week (Week 3). By evaluating autonomic nerve function using frequency analysis, it was found that SNA (LF/HF) decreases during inhalation of H₂, and paraSNA (HF/LF+HF) ratio increases in Week two compared to Week 1 (FIGS. 7a-d). The influence of H₂ gas in the autonomic nerve function disappeared after the first week after the end of inhalation.

### INDUSTRIAL APPLICABILITY

According to the pharmaceutical composition according to the present disclosure, the pharmaceutical composition according to the present disclosure can be used for improvement of a wide range of hypertension including essential hypertension.

1. A gaseous pharmaceutical composition for improving hypertension, characterized in that it comprises hydrogen gas.

2. The pharmaceutical composition according to claim 1, wherein the improvement of said hypertension comprises the improvement of diastolic hypertension.

3. The pharmaceutical composition according to claim 2, wherein a subject having said hypertension is a subject having a diastolic blood pressure of 100 mmHg or higher.

4. The pharmaceutical composition according to claim 1, wherein said hypertension is essential hypertension or secondary hypertension.

5. The pharmaceutical composition according to claim 4, wherein said hypertension is essential hypertension.

6. The pharmaceutical composition according to claim 4, wherein said hypertension is secondary hypertension.

7. The pharmaceutical composition according to claim 6, wherein said secondary hypertension is hypertension attributed to renal dysfunction.

8. The pharmaceutical composition according to claim 7, wherein said kidney dysfunction is attributed to renal disease or attributed to nephrectomy or partial nephrectomy.

9. The pharmaceutical composition according to claim 6, wherein a subject having said secondary hypertension is a subject that has not received dialysis.

10. The pharmaceutical composition according to claim 1, characterized in that said pharmaceutical composition further comprises oxygen gas.

11. The pharmaceutical composition according to claim 1, characterized in that said pharmaceutical composition further comprises inert gas.

12. The pharmaceutical composition according to claim 1, characterized in that said hydrogen concentration in said pharmaceutical composition is 0.1%-4.0% (v/v).

13. The pharmaceutical composition according to claim 1, characterized in that said hydrogen concentration in said pharmaceutical composition is 1.0%-2.0% (v/v).

14. The pharmaceutical composition according to claim 1, characterized in that said hydrogen gas is provided by a hydrogen gas generator or a container containing hydrogen gas.

15. The pharmaceutical composition according to claim 14, characterized in that said hydrogen gas generator is equipped with a hydrogen production means by water electrolysis.

16. The pharmaceutical composition according to claim 14, characterized in that said container contains a mixed gas of hydrogen gas and nitrogen gas.

17. The pharmaceutical composition according to claim 14, characterized in that said hydrogen gas generator or said container is further equipped with a control means for monitoring and adjusting the amount of hydrogen gas supplied to the subject on said device or container itself or on a piping connected to said device or container.

18. A method for hypertension therapy comprising administering hydrogen gas to a subject having hypertension.
